(19) United States
(12) Patent Application Publication
(10) Pub. No.: US 2009/0306441 A1
(43) Pub. Date: Dec. 10, 2009

(54) Method for treatment of oil and/or gas field waste and by product materials

(76) Inventors: Jean Marie Basset, Caluire (FR); Francois Stoffelbach, Lyon (FR); Mostafa Taoufik, Villeurbanne (FR); Jean Thivolle-Cazat, Fontaines sur Saone (FR)

Correspondence Address: NIXON & VANDERHYE, PC, 901 NORTH GLEBE ROAD, 11TH FLOOR, ARLINGTON, VA, 22203, US

(21) Appl. No.: 12/308,747

(22) PCT Filed: Jun. 12, 2007
(86) PCT No.: PCT/GB2007/002154

§ 371 Date: Dec. 23, 2008

(30) Foreign Application Priority Data
Jun. 26, 2006 (EP) 06253319.5

(51) Int. Cl. C10L 1/16; C07C 6/00

(52) U.S. Cl. 585/14; 585/708

(57) ABSTRACT

The present invention relates to a process for preparing 2,3-dimethylbutane which comprises contacting in a reaction zone isobutane with a supported catalyst comprising a tungsten hydride and a support comprising an aluminium oxide, so as to form a reaction mixture comprising 2,3-dimethylbutane. The contacting essentially leads to performing a metathesis reaction of the isobutane, with a very high specificity in the formation of 2,3-dimethylbutane. The catalyst is preferably a tungsten hydride grafted onto a support based on aluminium oxide. The support can be chosen from aluminium oxides, mixed aluminium oxides and modified aluminium oxides. The reaction mixture can be isolated and preferably subjected to one or more fractionating operations in order to recover 2,3-dimethylbutane and optionally one or more other components of the reaction mixture, such as C₅+ alkanes. The process can comprise isolating from the reaction mixture the C₅+ alkanes including 2,3-dimethylbutane as a single component, which can be blended with gasoline to enhance the gasoline octane number, or be used as a gasoline blendstock. At least one separated fraction containing 2,3-dimethylbutane can be isolated from the single component, which can be blended with gasoline to enhance the gasoline octane number, or be used as a gasoline blendstock.

The present invention relates to a process for preparing 2,3-dimethylbutane. 2,3-dimethylbutane, also called diisopropyl, is known to exhibit a high octane number, for example an RON (Research Octane Number) equal to 104, and a relatively low vapour pressure (51 kPa at 38° C.) (Internal Combustion Engines and Air Pollution, 1973, by E. F. Obert). For this reason, 2,3-dimethylbutane is sought as an additive to gasolines for the motor car, and it would be very useful to develop a method of preparing said product by a simple and direct process.

U.S. Pat. No. 4,255,605 describes a process for preparing 2,3-dimethyl butane from a mixed butenes feed stream comprising butene-1, butenes-2, isobutane, n-butane and isobutylene. The process comprises the steps of (a) subjecting the mixed butenes feed stream to double bond isomerization to convert butene-1 to butenes-2, (b) fractionating the effluent of step (a) into an overhead comprising isobutane, isobutylene and butene-1 and a bottoms stream comprising n-butane and butenes-2, (c) subjecting the bottoms stream in (b) to skeletal isomerization to convert butenes-2 to isobutylene, (d) combining the effluent from (c) with the effluent from (a) and fractionating the combined streams in (b), (e) disproportionating the overhead in step (b) to convert isobutylene to ethylene and 2,3-dimethylbutene-2, and butenes-2 to ethylene and normal hexene and heavier olefinic hydrocarbons, (f) fractionating the effluent from (e) into an overhead comprising C₂ and isobutane, a side stream comprising butylenes, and a bottoms stream comprising 6 carbon hydrocarbons including 2,3-dimethylbutene-2 and n-hexane and heavies, (g) recycling said side stream separated in (f) to step (e) for disproportionation, (h) hydrogenating said bottoms stream separated in (f) to produce n-hexane and 2,3-dimethylbutane, and (i) separating 2,3-dimethylbutane as product. However, the process is a long multiple-step process, and no supported catalyst comprising tungsten hydride and a support comprising an aluminium oxide is used.

International patent application WO 98/02244 describes a process for carrying out the metathesis of alkanes into their higher and lower homologues. It is thus possible to react an alkane with itself and to obtain directly its higher and lower homologues, more particularly in the presence of a supported catalyst comprising a metal hydride grafted and dispersed onto a solid oxide. The examples show that it is possible to use linear or branched alkanes such as ethane, propane, butane or isobutane, and various catalysts, such as a tantalum or tungsten hydride grafted onto a silica support. There is shown in particular an example of a metathesis reaction of isobutane (in static reactor) in the presence of a supported catalyst based on tantalum hydride grafted onto a silica. Said reaction forms a mixture of methane, ethane, propane, neopentane, isopentane and 2-methylpentane and, in smaller proportions, n-butane and 2-methylhexane. No mention was made of a formation of 2,3-dimethylbutane.

International patent application WO 2004/089541 describes a supported alkane metathesis catalyst comprising a tungsten hydride and a support based on aluminium oxide. It is shown that said catalyst used in hydrocarbon metathesis reactions exhibits a very high selectivity in the formation of linear (or normal) hydrocarbons (i.e. with linear chain) in comparison with the formation of branched hydrocarbons (i.e. with branched chain or in “iso” form). The examples show in particular metathesis of propane wherein essentially ethane and butanes were formed with low proportions of methane, pentanes and C6 homologues. The teaching of said application suggests that said catalyst would have a very low selectivity in the formation of branched alkanes prepared from linear alkanes; in particular, the formation of 2,3-dimethylbutane (exhibiting a double “iso” form) was not specifically mentioned. In addition, it was shown that this catalyst led to the formation mainly of alkanes immediately lower and higher than the starting alkane.

U.S. Pat. No. 6,441,263 and U.S. Pat. No. 6,566,569, the article by R. L. Burnett and T. R. Hughes in J. Catal., 1973, 31, 55-64, and the Article by A. S. Goldman, A. H. Roy, Z. Huang, R. Ahuja, W. Schinski and M. Brookhart in Science 2006, 312, 257-261 also describe reactions for the disproportionation of alkanes into their lower and higher homologues, but mainly leading to linear alkanes

It was found surprisingly that contrary to the teaching in particular of international patent application WO 2004/089541, the supported catalyst used in an isobutane metathesis reaction, comprising a tungsten hydride and a support based on aluminium oxide, exhibits a very high selectivity in the formation of 2,3-dimethylbutane. It was found in particular that said selectivity can be up to 3 times greater than that of a reaction that is identical but carried out in the presence of a supported catalyst comprising a tantalum hydride and a silica support. In addition, said result is all the more surprising in that the isobutane metathesis reaction ought to have led to the formation mainly of alkanes immediately higher or lower than isobutane, that is to say to C₅ and C₃ alkanes respectively, and not to C₆ and C₂ ones. It follows from this that such a reaction becomes an interesting route to a direct and simple preparation of 2,3-dimethylbutane, and said preparation forms the subject of the present invention.

The present invention relates to a process for the preparation of 2,3-dimethylbutane, characterised in that isobutane is contacted in a reaction zone with a supported catalyst comprising a tungsten hydride and a support comprising an aluminium oxide, so as to form a reaction mixture comprising 2,3-dimethylbutane.

The preparation of 2,3-dimethylbutane uses in particular a catalytic reaction for the metathesis of isobutane. The isobutane can be used alone or in the form of a mixture with one or more hydrocarbons. Preferably, the isobutane is used alone or substantially alone, and, in this case, the contacting according to the invention can lead mainly to a metathesis reaction of the isobutane with itself (i.e. an isobutane homologation reaction or an isobutane self-metathesis reaction). In such a reaction, the 2,3-dimethylbutane can be formed with a molar selectivity equal to or more than 25%, preferably equal to or more than 30%, in particular equal to or more than 40%. By molar selectivity to 2,3-dimethylbutane (expressed in %) is meant in general the ratio (multiplied by 100) of the number of moles of 2,3-dimethylbutane (2.3diMeBu) formed to the total number of moles of all the hydrocarbons formed, and which can be written according to the following equation (1):

Selectivity_{2.3diMeBu}=100×(number of 2,3diMeBu moles formed/total number of moles of all hydrocarbons formed) (1)

Similarly, and in a more general fashion, the molar selectivity to an alkane formed (expressed in %) corresponds to the ratio (multiplied by 100) of the number of moles of said alkane formed to the total number of moles of all the hydrocarbons formed.

The isobutane can also be used in the form of a mixture with one or more other hydrocarbon(s), preferably one or more other alkane(s), more particularly one or more other linear and/or branched alkane(s), in particular containing from 1 to 12 carbon atoms, for example from 4 to 12 carbon atoms, especially 4 carbon atoms. In said mixture, the isobutane can be, preferably, the majority molar constituent, representing for example from 50 to less than 100% of moles, or from 50 to 99% of moles of the mixture. It can also be a minority molar constituent, for example from 1 to less than 50% of moles, or from 5 to less than 50% of moles of the mixture. It is possible, for example, to use a mixture of isobutane with one or more other hydrocarbon(s), in particular one or more other alkane(s), having a proportion of isobutane such that after the contacting with the catalyst, the proportion of 2,3-dimethylbutane formed corresponds to that desired to obtain a gasoline for motor cars that has a desired octane number. Thus, in the case of a mixture of isobutane with one or more other hydrocarbon(s), the contacting according to the invention can lead simultaneously to a metathesis reaction of isobutane with itself (i.e. an isobutane self-metathesis reaction), reactions for the cross metathesis of isobutane with another hydrocarbon, metathesis reactions of a hydrocarbon with itself (i.e. hydrocarbon self-metathesis reactions), and reactions for the cross metathesis of a hydrocarbon with another hydrocarbon. Among said reactions, the reaction for the metathesis of isobutane with itself (i.e. an isobutane self-metathesis reaction) can be carried out according to the invention with a very high selectivity to 2,3-dimethylbutane.

The contacting of the isobutane is carried out in the presence of a supported catalyst comprising a tungsten hydride and a support comprising an aluminium oxide. It was found that in this case, said catalyst exhibits a very high selectivity for the formation of 2,3-dimethylbutane, in particular a selectivity such as that described above. The supported catalyst can comprise, preferably, a support based on aluminium oxide onto which is grafted a tungsten hydride. Thus, in this case, a tungsten atom or ion present in the catalyst can be bonded directly to the support comprising an aluminium oxide, more particularly to at least one oxygen atom of the aluminium oxide, in particular by a single tungsten-oxygen bond (W—OAl).

The catalyst comprises a support that can be any support comprising an aluminium oxide and more particularly any support where the aluminium oxide is directly accessible at the surface of the support. Thus, the support can be chosen preferably from aluminium oxide supports having in particular a homogeneous composition throughout their structure. It can also be chosen from heterogeneous aluminium oxide supports in which the aluminium oxide is mainly located at the surface of the support. In said latter case, the aluminium oxide can be dispersed, deposited, supported on or grafted onto a solid support that can itself be a support chosen more particularly from metallic or refractory oxides, sulphides, carbides, nitrides and salts, and from carbon, metals, open or closed mesoporous structures MCM21 and MCM22, organic/inorganic hybrid materials and molecular sieves, preferably chosen from silica and metallic or refractory oxides.

The support can have a specific surface area (B.E.T) measured according to the standard ISO 9277 (1995) which is chosen in a range of from 0.1 to 3000 m²/g, preferably from 0.1 to 1000 m²/g, preferably from 0.5 to 800 m²/g.

The support can be chosen from aluminium oxides, mixed aluminium oxides and modified aluminium oxides, more particularly modified by one or more elements of Groups 15 to 17 of the Periodic Table of the Elements. The Periodic Table of the Elements is that presented by IUPAC in 1991 in which the Groups are numbered from 1 to 18, and published by CRC Press, Inc., USA in “CRC Handbook of Chemistry and Physics” 76th edition (1995-1996), by David R. Lide.

The support can be chosen from aluminium oxides. By aluminium oxide, also called simple alumina, is understood generally an aluminium oxide substantially free of any other oxide, more particularly containing less than 2 wt % of one or more other oxide(s), that are generally present in the form of impurities. If it contains 2 wt % or more of one or more other oxide(s), it is generally agreed to consider the oxide a mixed aluminium oxide, more particularly in the form of an aluminium oxide combined with at least one other oxide.

The support is preferably chosen from aluminium oxides (or simple aluminas), in particular from porous aluminas, semi-porous aluminas, non-porous aluminas and mesoporous aluminas.

Thus, the support can be chosen from porous aluminas, often called “activated aluminas” or “transition aluminas”. They correspond generally to various partially hydroxylated aluminium oxides (Al₂O₃). They are generally obtained by an activation treatment comprising more particularly a thermal treatment (or dehydration treatment) of a precursor chosen for example from aluminium hydroxides such as aluminium trihydroxides, hydroxides of the aluminium oxide (or hydrates of the aluminium oxide) and gelatinous aluminium hydroxides (or alumina gels). The activation treatment makes it possible to remove the water contained in the precursor, and also in part the hydroxyl groups, thus allowing some residual hydroxyl groups and a porous structure to remain. Eventually, the porous structure may be avoided, when a flame alumina is used, and in this case the pre-treatment also removes the hydroxyl groups. The surface of the porous alumina comprises generally a complex mixture of atoms of aluminium and of oxygen, and also of hydroxyl ions that can combine according to specific crystalline forms and that can present both acidic and basic sites. The various crystalline forms depend generally on the choice of the precursor and the conditions of the activation treatment, such as the use of an air current or of another gas such as an inert gas, the pressure and the temperature, for example a temperature of from 100 to 1000° C., preferably from 200 to 1000° C. The support can be a porous alumina chosen more particularly from a γ-alumina (gamma alumina), a η-alumina (eta alumina), a δ-alumina (delta alumina), a θ-alumina (theta alumina), a κ-alumina (kappa alumina), a ρ-alumina (rho alumina), an α-alumina (alpha alumina) and a χ-alumina (ksi- or chi-alumina). It is preferred to choose the support from a γ-alumina and a η-alumina. The porous alumina can have a specific surface area (B.E.T.) of from 100 to 3000 m²/g, or from 100 to 1000 m²/g, preferably from 300 to 1000 m²/g, more particularly from 300 to 800 m²/g, in particular from 300 to 600 m²/g. It can also possess a specific pore volume equal to or less than 1.5 cm³/g, or equal to or less than 1 cm³/g, preferably equal to or less than 0.9 cm³/g, more particularly equal to or less than 0.6 cm³/g.

The support can also be chosen from the semi-porous aluminas. These are generally obtained by an activation treatment as described above, more particularly at a temperature ranging from 600 to 1000° C. They can comprise a mixture of a porous alumina, such as one of those described above, with a non-porous alumina, such as an α-alumina (alpha alumina) or a γ-alumina (gamma alumina), in ratios by weight between porous alumina and non-porous alumina that can range from 10/90 to 90/10, in particular from 20/80 to 80/20.

The support can also be chosen from the non-porous aluminas, known generally under the term “calcined alumina” or “flame alumina”, and which can be an α-alumina (alpha alumina) or a γ-alumina (gamma alumina). The α-alumina exists in the natural state under the name “corundum” and can contain impurities such as other oxides at the rate of 2 wt % or less, preferably of 1 wt % or less. It can also be synthesised, generally by a thermal treatment or a calcination of a precursor chosen more particularly from aluminium alkyls, aluminium salts, hydroxides of the aluminium oxide, aluminium trioxides and aluminium oxides, in particular at a temperature of more than 1000° C., preferably more than 1100° C. The non-porous aluminas can have a specific surface area (B.E.T.) ranging from 0.1 to 300 m²/g, preferably from 0.5 to 300 m²/g, more particularly from 0.5 to 250 m²/g.

The support can also be chosen from mesoporous aluminas, having more particularly a specific surface area (B.E.T.) ranging from 100 to 800 m²/g. They can have pores having a width ranging from 2 nm to 0.05 μm.

The support can be chosen from mixed aluminium oxides. By mixed aluminium oxide is meant generally an aluminium oxide combined with at least one other oxide in a proportion by weight that can range from 2 to less than 80%, more particularly from 2 to less than 50%, in particular from 2 to less than 40% or even from 2 to less than 30%. The other oxide or oxides can be oxides of the elements (M) chosen from the metals of Groups 1 to 13 and from the elements of Group 14, with the exception of carbon, of the Periodic Table of the Elements. The element (M) can be chosen from alkaline metals, alkaline-earth metals, transition metals, lanthanides and actinides, preferably chosen from silicon, boron, gallium, germanium, titanium, zirconium, cerium, vanadium, niobium, tantalum, chromium, molybdenum and tungsten. More particularly, the mixed aluminium oxides can be chosen from anhydrous aluminates, spinels, silica-aluminas and aluminosilicates.

The support can also be chosen from modified aluminium oxides, more particularly modified by one or more elements of Groups 13 to 17, preferably of Groups 15 to 17, preferably of Group 16 or 17, of the Periodic Table of the Elements. The aluminium oxides can be, in particular, modified by boron, phosphorus, sulphur, fluorine and/or chlorine. The support can be chosen more particularly from the super-acids of alumina, or from borated, boric, phosphated, pyrophosphated, phosphoric, orthophosphoric, phosphorous, orthophosphorous, sulphated, sulphurised, sulphuric, sulphurous, chlorinated or fluorinated oxides of aluminium, preferably from chlorinated oxides of aluminium.

The support can be in the form of particles that can have any shape and any size. The particles can have a mean size of from 10 nm to 10 mm or from 10 nm to 5 mm, preferably from 20 nm to 4 mm. They can have a spherical, spheroidal, hemispherical, hemispheroidal, cylindrical or cubic shape, or a ring, pellet, disc or granule shape, or else a shape of packing materials such as those used in a distillation column reactor, as described in U.S. Pat. No. 4,242,530.

The supported catalyst comprises a tungsten hydride and a support comprising an aluminium oxide onto which the tungsten hydride is preferably grafted. Tungsten can have an oxidation state in a range of from 2 to 6, preferably from 4 to 6. Tungsten atoms (or ions) present in the supported catalyst can be bonded to the support more particularly by at least one single bond, as well as to one or more hydrogen atoms more particularly by single bonds (W—H), and optionally to one or more hydrocarbon radicals, R, more particularly by single or multiple carbon-tungsten bonds. The number of hydrogen atoms bonded to tungsten can be from 1 to 5, preferably from 1 to 4, more particularly from 1 to 3. By tungsten hydride grafted to the support is meant generally that the tungsten atom is bonded to the support by at least one single bond, more particularly to at least one oxygen atom of the aluminium oxide, for example by at least one single bond (W—OAl). Tungsten can also be bonded to one or more hydrocarbon radicals, R, more particularly by one or more single, double or triple carbon-tungsten bonds. The radical R can be chosen from the radicals methyl, ethyl, n-propyl, isopropyl, n-butyl, isobutyl, neopentyl, allyl, neopentylidene, allylidene, neopentylidyne and neosilyl. Tungsten can also be complexed by one or more hydrocarbon ligands, in particular aromatic ligands, and/or by one or more carbonyl ligands.

The supported catalyst is a tungsten hydride as described above, which also can comprise one or more ligands, such as “ancillary” ligands, preferably comprising at least one oxygen atom and/or at least one nitrogen atom. The ligands can be identical or different, and can be preferably chosen from oxo, alkoxo, aryloxo, aralkyloxo, nitrido, imido and amido ligands. There is meant generally by oxo, alkoxo, aryloxo, aralkyloxo, nitrido, imido and amido ligands respectively:

a divalent oxo radical with the general formula: =O

a monovalent alkoxo, aryloxo or aralkyloxo radical with the general formula: —OR′

a trivalent nitrido radical with the general formula: =N

a divalent imido radical with the general formula: =R″, and

a monovalent amido radical with the general formula: —NR¹R²,

in which formulae O represents an oxygen atom, R′ represents a hydrogen atom or a monovalent hydrocarbon radical, linear or branched, saturated or unsaturated, more particularly selected respectively from alkyl radicals preferably from C₁ to C₁₀ for the alkoxo ligands, from aryl radicals preferably from C₆ to C₁₂ for the aryloxo ligands, and from aralkyl radicals preferably from C₇ to C₁₄ for the aralkyloxo ligands, N represents a nitrogen atom, R″ represents a hydrogen atom or a monovalent hydrocarbon radical, linear or branched, saturated or unsaturated, more particularly selected from alkyl radicals preferably from C₁ to C₁₀, from aryl radicals from C₆ to C₁₂, and from aralkyl radicals from C₇ to C₁₄, and R¹ and R² being identical or different represent a hydrogen atom or a monovalent hydrocarbon radical, linear or branched, saturated or unsaturated, more particularly selected from alkyl radicals preferably from C₁ to C₁₀, from aryl radicals preferably from C₆ to C₁₂, and from aralkyl radicals preferably from C₇ to C₁₄.

The catalyst exhibits generally in infrared spectroscopy one or more absorption bands specific for the (W—H) bond, bands whose frequency can vary according to the coordination sphere of the tungsten and can depend on the number of bonds of the tungsten with the support and with optionally the hydrocarbon radicals R and other hydrogen atoms. Thus, for example, at least two absorption bands were found at 1903 and 1804 cm⁻¹, bands specific for the (W—H) bond considered in the environment of the (W—OAl) bonds linking the same tungsten atom to an oxygen atom itself linked to an aluminium atom, more particularly in an α-alumina or a γ-alumina. It is also possible to characterise the (W—H) bond in the catalyst by NMR of the proton under 500 MHz where the value of the chemical shift of the tungsten hydride (δ_{W—H}) can vary and depends on the co-ordination sphere of the tungsten and on the number of bonds of the tungsten with the support and optionally with the hydrocarbon radicals R. In some typical cases, it may be equal to 0.6 ppm (parts per million).

As an example, the catalyst and its preparation are described more particularly in International patent application WO 2004/089541. The preparation of the catalyst can comprise the following stages:
(1) a stage of calcination under air or oxygen of the support comprising an aluminium oxide, for example of the α- or γ-alumina, for a period more particularly of 1 to 24 hours, preferably at a temperature of 200 to 1000° C., in particular of 300 to 700° C., followed by a stage involving dehydroxylation, for example under an atmosphere of an inert gas or under vacuum, more particularly for a period of 1 to 4 hours, preferably at a temperature of 200 to 1000° C., in particular of 300 to 700° C.,
(2) a stage of dispersion and grafting of an organometallic precursor (Pr) of tungsten onto a support based on aluminium oxide, in which precursor the tungsten can be bonded or complexed to at least one hydrocarbon ligand, so as to form a tungsten hydrocarbon compound or complex grafted onto the support, followed by
(3) a stage of hydrogenolysis of the preceding compound or complex, so as to form a tungsten hydride grafted onto the support.

The process for preparing 2,3-dimethylbutane comprises the contacting of isobutane with the supported catalyst comprising the tungsten hydride and a support comprising an aluminium oxide. The contacting can be carried out in various ways, more particularly at a temperature of from 50 to 600° C., preferably from 70 to 550° C., in particular from 100 to 500° C. It can also be performed under a total absolute pressure that can range from 0.01 to 100 MPa, preferably from 0.1 to 50 MPa, in particular from 0.1 to 30 MPa.

The contacting can also be carried out in the presence of an inert agent, either liquid or gaseous, in particular of an inert gas such as nitrogen, helium or argon. It can be advantageously performed in the presence of hydrogen or of an agent forming hydrogen “in situ”, such as a cyclic hydrocarbon chosen particularly from cyclohexane, decahydronaphtalene and tetrahydronaphtalene. The hydrogen present during the contacting can play the role of an agent of activation or regeneration for the catalyst. For example, hydrogen can be used in the contacting with a hydrogen partial pressure chosen in a wide range, preferably from 0.1 kPa to 50 MPa, in particular from 1 kPa to 1 MPa, or from 0.01 to 50 MPa, particularly from 0.1 to 20 MPa.

In addition, the contacting can be carried out with quantities of isobutane and catalyst such that the molar ratio of isobutane to tungsten of the catalyst is chosen in a wide range, for instance from 1 to 10⁷, preferably from 2 to 10⁵, in particular from 5 to 10⁴. It can also be performed in the reaction zone containing the catalyst and into which isobutane is introduced preferably continuously, in particular with a molar rate of introduction of isobutane per mole of tungsten of the catalyst and per minute which can be chosen in a very wide range, e.g. from 0.01 to 10⁵ or from 1 to 10⁵, or else from 5 to 10⁵, or from 0.01 to 10³, preferably from 0.1 to 5×10², more particularly from 0.5 to 10² or.

The contacting is performed in a reaction zone so as to form a reaction mixture essentially comprising 2,3-dimethylbutane and generally ethane, preferably in predominant proportions, with optionally unreacted isobutane. The reaction mixture can also comprise, but in lower proportions, methane, propane and other heavier alkanes, generally C₅₊ alkanes (i.e. comprising at least 5 carbon atoms), more particularly C₅ to C₈ alkanes, such as isopentane and linear and/or branched hexanes, heptanes and octanes. On the other hand, ethane and propane can also be separated and isolated from the reaction zone, and optionally subjected to other operations, such as a cracking in order to prepare olefins. Furthermore, the other heavier alkanes, more particularly C₅ to C₈ alkanes, in particular linear and/or preferably branched hexanes, heptanes and octanes, can likewise be either separated and isolated from the reaction zone and be used preferably and directly as additives with a high octane number for gasoline (e.g. for cars), or be maintained in mixture with 2,3-dimethylbutane and be used as a mixture of additives with a high octane number for gasoline (e.g. for cars). The entire (normally liquid) reaction mixture, after the removal of gaseous products, e.g. methane, ethane and propane, may also be used directly as a blending component to manufacture gasoline (e.g. for cars).

Various methods can be used for performing the contacting and improving the yield of the process. The contacting can be conducted discontinuously or preferably continuously. It can be performed in a gaseous phase, or in a mixed gaseous/liquid phase, or in a liquid phase, or else in a supercritical phase, in a reaction zone adapted to the phase chosen. Thus, the contacting can be performed in a gaseous or mixed gaseous/liquid phase by contacting gaseous isobutane over the catalyst and forming 2,3-dimethylbutane in a gaseous form or in a liquid form. The contacting can also take place in a liquid phase or in a supercritical phase by using liquid isobutane with the catalyst in suspension.

The contacting can be carried out in a reaction zone comprising a static reactor, a recycling reactor or a dynamic continuous flow reactor. In a static reactor, the reactor may contain fixed quantities of isobutane and catalyst, e.g. introduced for a complete reaction cycle. In a recycling reactor, it is preferred to recycle at least one of the components of the reaction mixture, preferably the unreacted isobutane and/or the 2,3-dimethylbutane formed. In a dynamic continuous flow reactor, the liquid or gaseous isobutane may be more particularly passed through a bed comprising the catalyst.

In practice, the contacting can be performed in a reaction zone comprising a reactor chosen from tubular (or multi-tubular) reactors, distillation column reactors, slurry reactors, fluidised bed reactors, mechanically agitated bed reactors, fluidised and mechanically agitated bed reactors, fixed bed reactors and circulating bed reactors. The catalyst, generally in particle form, can be arranged inside the tube(s) of a tubular (or multi-tubular) reactor. Thus, the isobutane introduced preferably continuously into the tube(s) can pass through it (or them) in the form of a stream and thus be contacted with the catalyst, so as to form the reaction mixture. The catalyst can also be arranged inside a distillation column reactor, wherein the catalyst is preferably a component of a distillation system functioning as both a catalyst and a distillation packing, i.e. a packing for a distillation column having both a distillation function and a catalytic function: for example, rings, saddles, granulates, sheets, tubes, spirals, packed in bags, as described in U.S. Pat. No. 4,242,530. The catalyst can also form the bed of a fluidised and/or mechanically agitated bed reactor, of a fixed bed reactor, or of a circulating bed reactor. The catalyst can be used in one of said reactors, optionally in mixture with at least one inert solid agent, preferably chosen from silicas, aluminas, silica-aluminas and aluminium silicates. The isobutane can be introduced into one of said reactors preferably continuously, and generally can pass or circulate preferably continuously in the form of a gaseous or liquid stream into the tube(s) or through the bed or the distillation packing of said reactors containing the catalyst. In order to promote the development of the reaction towards an optimum production of 2,3-dimethylbutane, the process can be advantageously performed by withdrawing preferably continuously one or more component(s) of the reaction mixture, preferably 2,3-dimethylbutane.

The reaction mixture thus formed in the reaction zone can be treated for separating and recovering 2,3-dimethylbutane from said reaction mixture. The reaction mixture which generally comprises 2,3-dimethylbutane and ethane with unreacted isobutane, can be also treated for separating the unreacted isobutane from said reaction mixture, while the unreacted isobutane thus separated is preferably returned into the reaction zone. More specifically, the reaction mixture comprising 2,3-dimethyl butane and generally ethane with optionally unreacted isobutane can be isolated from the reaction zone and preferably subjected to one or more fractionating operations, more particularly selected from distillation or change of liquid/gaseous phase, so as to isolate and to recover the 2,3-dimethylbutane and optionally the unreacted isobutane which is preferably returned into the reaction zone.

The reaction mixture which essentially comprises 2,3-dimethylbutane and generally ethane, particularly in predominant proportions, with optionally unreacted isobutane, can also comprise, but in lower proportions, methane, propane and other heavier alkanes, generally C₅₊ alkanes, more particularly C₅ to C₈ alkanes, such as isopentane, linear and/or preferably branched hexanes, heptanes and octanes. Thus, the process can comprise separating and isolating 2,3-dimethylbutane and optionally one or more other component(s) of the reaction mixture, separately or in mixture. The separation can be performed in various ways, discontinuously or preferably continuously. It can comprise one or more fractionation(s) of the reaction mixture, of an identical or different type, and preferably chosen from:

fractionation by change of physical state, preferably by change of gaseous/liquid phase, particularly by distillation and/or condensation or partial condensation, e.g. by means of distillation/condensation column or column reactor,

fractionation by molecular filtration, preferably by means of semi-permeable and selective membrane,

fractionation by adsorption, preferably by means of molecular sieve or any other adsorbent,

fractionation by absorption, preferably by means of absorbing oil,

fractionation by cryogenic expansion, preferably by means of expansion turbine, and

fractionation by compression, preferably by means of gas compressor.

Among these fractionations, the fractionation by change of the physical state of the reaction mixture, preferably by change of gaseous/liquid phase, particularly by distillation and/or condensation or partial condensation, in particular by means of one or more distillation/condensation column(s) or a distillation column reactor, is preferred.

The process can advantageously comprise separating and isolating from the reaction mixture the C₅₊ alkanes, more particularly the C₅ to C₈ alkanes (e.g. linear and/or preferably branched pentanes, hexanes, heptanes and octanes), including 2,3-dimethylbutane as a single component, so as preferably to blend said single component with gasoline in particular to enhance the gasoline octane number, or to use said single component as a gasoline blendstock.

The present invention also relates to the use of the previously mentioned single component comprising 2,3-dimethylbutane for blending it with gasoline preferably to enhance the gasoline octane number. It also relates to the use of said single component comprising 2,3-dimethylbutane, as a gasoline blendstock.

The process can also advantageously comprise separating from the reaction mixture the C₅₊ alkanes, more particularly the C₅ to C₈ alkanes, including 2,3-dimethylbutane as a single component, followed by separating and isolating at least one separated fraction comprising 2,3-dimethylbutane from said single component, so as preferably to blend said at least one separated fraction with gasoline preferably to enhance the gasoline octane number, or to use said at least one separated fraction as a gasoline blendstock.

The present invention also relates to the use of the previously mentioned at least one separated fraction comprising 2,3-dimethylbutane, for blending it with gasoline preferably to enhance the gasoline octane number. It also relates to the use of said separated fraction comprising 2,3-dimethylbutane, as a gasoline blendstock.

The process of the invention is also particularly advantageous for manufacturing 2,3-dimethylbutane, namely in a single (reaction) stage and with a relatively high specificity.

The following examples illustrate the present invention.

### EXAMPLE 1

### Preparation of a Catalyst Comprising a Tungsten Hydride Grafted onto a Support Based on Aluminium Oxide

2.5 g of a γ-alumina (Aeroxide® Alu C) having a specific surface (B.E.T.) of 100 m²/g and containing 94.95 wt % of alumina and 5 wt % of water, sold by Degussa (Germany), were subjected to a calcination treatment under a dry air current at 500° C. for 15 hours, followed by a dehydroxylation treatment under an absolute pressure of 10⁻² Pa, at 500° C. for 15 hours. The alumina thus treated exhibited in infrared spectroscopy three absorption bands, at 3774, 3727 and 3683 cm⁻¹ respectively, which are characteristic of residual (AlO—H) bonds.

In a first stage, 1.8 g of the alumina prepared beforehand were isolated and introduced under argon atmosphere into a glass reactor at 25° C. which was fitted with a magnetic stirring rod. There were then introduced into the reactor 305 mg of tris(neopentyl)neopentylidene tungsten, used as a precursor (Pr) of the catalyst, and corresponding to general formula (2):

W [—CH₂—C(CH₃)₃)]₃ [≡C—C(CH₃)₃] (2)

The reactor was heated to 66° C. and the mixture thus produced was agitated in the dry state for 4 hours. At the end of this time, the reactor was cooled to 25° C., after which the solids mixture was washed with n-pentane at 25° C. The solid compound washed in this way was vacuum dried, then isolated under argon so as to obtain an organometallic tungsten compound grafted onto the alumina, which contained 4.2 wt % of tungsten and corresponded to general formula (3):

(Al—O)ₓW [—CH₂—C(CH₃)₃]_{y} [≡C—C(CH₃)] (3)

with x=1 and y=2.

500 mg of the grafted organometallic tungsten compound obtained above were placed in a glass reactor with a capacity of 500 ml for a hydrogenolysis treatment performed by contacting with hydrogen, under an absolute hydrogen pressure of 73 kPa, at 150° C., for 15 hours. At the end of this period, the reactor was cooled to 25° C., and there was obtained and isolated under argon, and at atmospheric pressure, the catalyst (W—H/Al) comprising the tungsten hydride grafted onto alumina. The catalyst contained 4.2 wt % of tungsten and exhibited in infrared spectroscopy two absorption bands, at 1903 and 1804 cm⁻¹ respectively, that were characteristic of the (W—H) bond grafted onto the alumina. In addition, it exhibited in nuclear magnetic resonance (¹-H-NMR solid) under 500 MHz a value for the chemical shift of the tungsten hydride (δ_{W—H}) of 0.6 ppm (parts per million).

### EXAMPLE 2 (COMPARATIVE)

### Preparation of a Catalyst Comprising a Tantalum Hydride Grafted onto a Support Based on Silica

1.8 g of a silica sold under the commercial reference “Aerosil 200”® by Degussa (Germany), having a specific surface (B.E.T.) of 200 m²/g, were subjected to a dehydroxylation treatment under an absolute pressure of 10⁻² Pa, at 500° C. for 15 hours. A silica exhibiting in infrared spectroscopy an absorption band at 3747 cm⁻¹, which was characteristic of the residual (SiO—H) bond, was thus obtained.

1.4 g of the silica prepared above was introduced into a glass reactor under an argon atmosphere at 25° C. There was then introduced into the reactor a quantity of 15 ml of n-pentane containing 270 mg of tris(neopentyl)neopentylidene tantalum, used as a precursor (Pr) of the catalyst, and corresponding to general formula (4):

Ta [—CH₂—C(CH₃)₃]₃ [═CH —C(CH₃)₃] (4)

The mixture thus obtained was maintained at 25° C. for 2 hours, so as to obtain an organometallic tantalum compound grafted onto silica. At the end of this period, the excess of unreacted precursor (Pr) was removed, by washing with n-pentane at 25° C. The organometallic tantalum compound thus grafted was vacuum dried. It contained 5.2 wt % of tantalum and corresponded to general formulas (5) and (6):

(Si—O)ₓTa [—CH₂—C(CH₃)₃]_{y} [═CH —C(CH₃)₃]

with x=1, y=2 (5)

and with x=2, y=1. (6)

The organometallic tantalum compound grafted onto silica thus prepared was subjected to a hydrogenolysis treatment performed by contacting with hydrogen, under an absolute hydrogen pressure of 73 kPa, at 150° C. for 15 hours. At the end of this period, the catalyst comprising a tantalum hydride grafted onto silica (Ta—H/Si) was obtained and isolated under argon. It contained 5.2 wt % of tantalum and exhibited under infrared spectroscopy an absorption band at 1830 cm⁻¹ which was characteristic of the (Ta—H) bond grafted onto silica.

### EXAMPLE 3

### Preparation of 2,3-dimethylbutane

The preparation of the 2,3-dimethylbutane was performed in the following manner: Isobutane was introduced continuously at a rate of 4 ml/min, under an overall absolute pressure of 0.1 MPa, across a reactor with a capacity of 5 ml which was heated to 150° C. and which contained 500 mg of the catalyst comprising tungsten hydride grafted to alumina (W—H/Al), prepared in Example 1.

It was observed that the reaction mixture formed by the contacting predominantly contained 2,3-dimethylbutane and ethane coming from isobutane homologation reaction in the presence of the catalyst (W—H/Al), according to the following main equation (7):

2CH(CH₃)₃→CH(CH₃)₂—CH(CH₃)₂+CH₃—CH₃ (7)

In the reaction mixture formed, there were also found, but in small proportions, methane, propane, isopentane and other alkanes, particularly C₅₊ alkanes, such as C₅ to C₈ alkanes, namely linear and branched pentanes, hexanes, heptanes and octanes.

The molar selectivity in the formation of 2,3-dimethylbutane, which was equal more particularly to 41.2% (after 600 minutes of reaction), and the molar selectivities of other alkanes formed were measured (see Table 1 below).

### EXAMPLE 4 (COMPARATIVE)

### Preparation of 2,3-dimethylbutane

Exactly the same procedure was adopted as in Example 3, except that use was made of 330 mg of the catalyst comprising the tantalum hydride grafted onto silica (Ta—H/Si) prepared in Example 2 (comparative) in place of the 500 mg of the catalyst (W—H/Al).

It was observed that the reaction mixture formed by the contacting predominantly contained isopentane and ethane, and also, but in lower proportions, propane, 2,3-dimethylbutane, methane and other alkanes. The homologation reaction of the isobutane in the presence of the catalyst comprising tantalum hydride grafted onto silica (Ta—H/Al) can be written according to the following main equations (8) and (9):

2CH(CH₃)₃→CH₃—CH₂—CH₃+CH₃—CH(CH₃)—CH₂—CH₃ (8)

2CH(CH₃)₃→CH₃—CH₃+CH(CH₃)₂—CH(CH₃)₂ (9)

The molar selectivity in the formation of 2,3-dimethylbutane, which was equal more particularly to 15.5% (after 600 minutes of reaction), and the molar selectivities of other alkanes formed were measured for comparison purposes (see Table 1 below).

It was noted, when analysing Table 1, that the molar selectivity to 2,3-dimethylbutane was of the order of 41% in the reaction of Example 3 according to the present invention, while it was of the order of only 15% in the reaction of Example 4 (comparative).

Alkane (linear as well as branched chain) metathesis reactions carried out in the presence of metal hydride catalysts are known from prior art to produce predominantly linear alkanes as the main reaction products. In addition, based on prior art, it was expected that the reaction products of the alkane metathesis are mainly alkanes having carbon numbers one immediately lower and one immediately higher than the starting alkane. The results shown in Table 1 were surprising in that the isobutane metathesis reaction ought to have led to the formation mainly of alkanes immediately lower and higher than isobutane, i.e. of C₃ and C₅ alkanes respectively, as shown by Example 4 comparative. Surprisingly, in Example 3 according to the present invention, C₂ and C₆ alkanes, especially 2,3-dimethylbutane with a high specificity, were predominantly obtained.

TABLE 1
Molar selectivities (in %) of various alkanes obtained (for
100 moles of all the hydrocarbons formed) in Examples 3 and
4 (comparative), after 600 and 2500 minutes of reaction.

| | Molar selectivity (%) | | | |
|---|---|---|---|---|
| | | | Ta—H/Si (Example 4 | |
| | W—H/Al (Example 3) | | comparative) | |
| | after | after | after | after |
| Product | 600 min | 2500 min | 600 min | 2500 min |
| CH₄ | 2.6 | 1.4 | 8.4 | 7.3 |
| C₂H₆ | 40.5 | 40.8 | 27.5 | 28.2 |
| C₃H₈ | 6.7 | 4.8 | 16.1 | 14.6 |
| C₅H₁₂ total | 5.9 | 5.3 | 23.1 | 21.1 |
| isopentane | 5.8 | 5.2 | 22.3 | 19.6 |
| C₆H₁₄ total | 41.6 | 42.4 | 17.8 | 18.5 |
| 2,3- | 41.2 | 41.3 | 15.5 | 15.8 |
| dimethylbutane | | | | |
| C₇H₁₆ | 0.8 | 1.0 | 4.2 | 4.0 |
| C₈H₁₈ | 0.6 | 1.0 | 1.2 | 1.3 |

1. Process preparing 2,3-dimethylbutane, characterised in that isobutane is contacted in a reaction zone with a supported catalyst comprising a tungsten hydride and a support comprising an aluminium oxide, so as to form a reaction mixture comprising 2,3-dimethylbutane.

2. Process according to claim 1, characterised in that the isobutane is used alone or in the form of a mixture with one or more other hydrocarbon(s).

3. Process according to claim 2, characterised in that the isobutane is used in the form of a mixture with one or more other alkane(s).

4. Process according to claim 2, characterised in that the isobutane is used in the form of a mixture with one or more other linear and/or branched alkane(s).

5. Process according to claim 1, characterised in that the catalyst comprises a support based on aluminium oxide onto which is grafted a tungsten hydride.

6. Process according to claim 1, characterised in that the support is chosen from aluminium oxides, mixed aluminium oxides and modified aluminium oxides.

7. Process according to claim 6, characterised in that the modified aluminium oxides comprise one or more elements of Groups 13 to 17 of the Periodic Table of the Elements.

8. Process according to claim 1, characterised in that the support has a specific surface area (B.E.T.) chosen from a range of from 0.1 to 3000 m²/g, preferably

9. Process according to claim 1, characterised in that the support is chosen from porous aluminas, semi-porous aluminas, non-porous aluminas and mesoporous aluminas.

10. Process according to claim 1, characterised in that the contacting is performed at a temperature chosen in a range of from 50 to 600° C., preferably from 70 to 550° C.

11. Process according to claim 1, characterised in that the contacting is performed under a total absolute pressure chosen in a range of from 0.01 to 100 MPa, preferably from 0.1 to 50 MPa.

12. Process according to claim 1, characterised in that the contacting is performed in the presence of hydrogen or an agent forming hydrogen in situ, preferably under a hydrogen partial pressure chosen from 0.1 kPa to 50 MPa, or from 0.01 to 50 MPa.

13. Process according to claim 1, characterised in that the contacting is performed with quantities of isobutane and catalyst such that the molar ratio of isobutane to tungsten of the catalyst is chosen from 1 to 10⁷, preferably from 2 to 10⁵.

14. Process according to claim 1, characterised in that the contacting is performed in the reaction zone containing the catalyst and into which isobutane is introduced preferably continuously with a molar rate of introduction of isobutane per mole of tungsten of the catalyst and per minute, chosen from 0.01 to 10⁵, preferably from 0.01 to 10³, more particularly from 0.1 to 5×10².

15. Process according to claim 1, characterised in that the contacting is performed in a gaseous phase, in a mixed gaseous/liquid phase, in a liquid phase or in a supercritical phase.

16. Process according to claim 1, characterised in that the reaction zone comprises a static reactor, a recycling reactor or in a dynamic continuous flow reactor.

17. Process according to claim 1, characterised in that the reaction zone comprises a reactor chosen from tubular (or multi-tubular) reactors, distillation column reactors, slurry reactors, fluidised bed reactors, mechanically agitated bed reactors, fluidised and mechanically agitated bed reactors, fixed bed reactors and circulating bed reactors.

18. Process according to claim 1, characterised in that the process comprises separating and isolating 2,3-dimethylbutane and optionally one or more other components) of the reaction mixture, separately or in mixture.

19. Process according to claim 18, characterised in that separation is performed discontinuously or preferably continuously, and comprises one or more fractionation(s) of the reaction mixture, of an identical or different type, and preferably chosen from:
fractionation by change of physical state, preferably by change of gaseous/liquid phase, particularly by distillation and/or condensation or partial condensation, in particular by means of distillation/condensation column or column reactor,
fractionation by molecular filtration, preferably by means of semi-permeable and selective membrane,
fractionation by adsorption, preferably by means of molecular sieve or any other adsorbent,
fractionation by absorption, preferably by means of absorbing oil;
fractionation by cryogenic expansion, preferably by means of expansion turbine, and
fractionation by compression, preferably by means of gas compressor.

20. Process according to claim 1, characterised in that a reaction mixture comprising 2,3-dimethylbutane and ethane with optionally unreacted isobutane is formed by the contacting in the reaction zone and is treated for separating and recovering the 2,3-dimethylbutane from said reaction mixture.

21. Process according to claim 1, characterised in that a reaction mixture comprising 2,3-dimethylbutane and ethane with unreacted isobutane is formed by the contacting in the reaction zone and is treated for separating the unreacted isobutane from said reaction mixture, while the unreacted isobutane thus separated is returned into said reaction zone.

22. Process according to claim 1, characterised in that a reaction mixture comprising 2,3-dimethylbutane and ethane with optionally unreacted isobutane is formed by the contacting in the reaction zone, and is isolated from said zone.

23. Process according to claim 1, characterised in that a reaction mixture comprising 2,3-dimethylbutane and ethane with optionally unreacted isobutane is formed by the contacting in the reaction zone, is isolated from said zone and is subjected to one or more fractionating operation(s) selected from distillation and change of liquid/gaseous phase, so as to isolate and to recover the 2,3-dimethylbutane and optionally the unreacted isobutane.

24. Process according to claim 23, characterised in that the unreacted isobutane isolated and recovered by the fractionating operation(s) is returned into the reaction zone.

25. Process according to claim 1, characterised in that the contacting forms a reaction mixture comprising C₅₊ alkanes, preferably C₅ to Cs alkanes, including 2,3-dimethylbutane, and the process comprises separating and isolating from the reaction mixture said C₅₊ alkanes, preferably said Cs to Cs alkanes, including 2,3-dimethylbutane as a single component, so as preferably to blend said single component with gasoline in particular to enhance the gasoline octane number, or to use said single component as a gasoline blendstock.

26. Process according to claim 1, characterised in that the contacting forms a reaction mixture comprising C₅₊ alkanes, preferably C₅ to Cs alkanes, including 2,3-dimethylbutane, and the process comprises separating from the reaction mixture said C₅₊ alkanes, preferably C₅ to Cg alkanes, including 2,3-dimethylbutane as a single component, followed by separating and isolating at least one separated fraction containing 2,3-dimethylbutane from said single component, so as preferably to blend said at least one separated fraction with gasoline in particular to enhance’ the gasoline octane number, or to use said at least one separated fraction as a gasoline blendstock.

27. Use of the single component comprising 2,3-dimethylbutane according to claim 25, for blending said single component with gasoline, preferably to enhance the gasoline octane number.

28. Use of the single component comprising 2,3-dimethylbutane according to claim 25, as a gasoline blendstock.

29. Use of at least one separated fraction comprising 2,3-dimethylbutane according to claim 26, for blending said at least one separated fraction with gasoline, preferably to enhance the gasoline octane number.

30. Use of at least one separated fraction comprising 2,3-dimethylbutane according to claim 26, as a gasoline blendstock.
